(19) United States
(12) Patent Application Publication
(10) Pub. No.: US 2021/0393662 A1
(43) Pub. Date: Dec. 23, 2021

(54) USE OF SGLT2 INHIBITORS TO TREAT PRIMARY SCLEROSING CHOLANGITIS

(71) Applicant: AVOLYNT, Research Triangle Park, NC (US)

(72) Inventors: William Owen WILKISON, Raleigh, NC (US); James Trinca GREEN, Raleigh, NC (US)

(21) Appl. No.: 17/285,675

(22) PCT Filed: Oct. 17, 2019
(86) PCT No.: PCT/US2019/056658

§ 371 Date: Apr. 15, 2021

Related U.S. Application Data
(60) Provisional application No. 62/747,363, filed on Oct. 18, 2018.

(51) Int. Cl. A61K 31/7056; A61P 1/16

(52) U.S. Cl. CPC A61K 31/7056; A61P 1/16

(57) ABSTRACT

The invention relates to the use of pharmaceutical compositions of the SGLT2 inhibitor, remogliflozin etabonate, to treat primary sclerosing cholangitis (PSC). Methods and compositions associated with the invention can improve or maintain clinical outcomes of PSC symptoms, such as ascites accumulation, hepatic encephalopathy, development of varices, jaundice, variceal bleeding, cholangiocarcinoma, hepatocellular carcinoma, evidence of cirrhosis, and colorectal cancer.

### FIELD OF THE INVENTION

The invention relates to compositions and methods associated with using an inhibitor of the sodium/glucose transporter 2 (“SGLT2”) to treat primary sclerosing cholangitis (“PSC”).

### BACKGROUND

Primary sclerosing cholangitis (PSC) is a serious, chronic cholestatic liver disease characterized by a progressive, autoimmune-based destruction of the bile duct, and the eventual onset of cirrhosis and its complications, though PSC symptoms may remain quiescent for long periods of time in some patients. Remissions and relapses characterize the disease course. While the cause of PSC is unknown, it is believed that damage to the bile duct occurs through one or more of genetic abnormalities of immune regulation, viral infection, toxins from intestinal bacteria, bacteria in the portal venous system, ischemic vascular damage, and toxic bile acids from intestinal bacteria. One particular immune regulation abnormality that conveys an increased risk of developing PSC, is hyper-IgM syndrome, a disorder characterized by lack of IgG and IgA due to deficient immunoglobulin class-switching. The majority of patients with PSC also have an underlying inflammatory bowel disease (“IBD”), typically ulcerative colitis (“UC”) or Crohn's disease. Among the foregoing PSC patients with IBD, 85% have UC, and 15% have Crohn's disease. Overall, 2.5-7.5% of all UC patients have PSC. PSC patients are also at an increased risk for cholangiocarcinoma, with 10-15% of the PSC patient population eventually developing this disorder. The pathogenesis of PSC is unclear, but it most frequently occurs as a complication of UC in humans, suggesting some overlap in pathogenetic mechanisms.

PSC is usually diagnosed by preliminary assessment of liver biochemistry, with or without reported symptoms, and confirmed by cholangiography, typically magnetic resonance cholangiopancreatography or endoscopic retrograde cholangiopancreatography (“ERCP”). Elevated alkaline phosphatase (“ALP”) activity is common in most PSC patients, and consistent with cholestasis. Alanine aminotransferase (“ALT”) and gamma glutamyltransferase (“GGT”) are also typically elevated in PSC patients, but not in all cases. Bilirubin levels are often normal in early-stage PSC, but increase with disease progression. The mean age at diagnosis is approximately 40 years, and the median time period of survival for PSC patients has been estimated as 8 to 12 years, from diagnosis in symptomatic patients, depending upon stage of the disease at the time of diagnosis. Complications involving the biliary tree are common and include cholangitis as well as ductal strictures and gallstones, both of which may require frequent endoscopic or surgical interventions. PSC is also often complicated by the development of malignancies, with cholangiocarcinoma being the most common.

At the organ level, PSC is a chronic fibrosing inflammatory process in the liver, which results in the destruction of the biliary tree and biliary cirrhosis. Biliary strictures are located in both the intrahepatic and extrahepatic ducts in more than 80% of the patients, but about 10% of these patients have only intrahepatic strictures, while less than 5% will have only extrahepatic strictures. The most specific histologic finding in humans with PSC is concentric “onion skin” fibrosis of small interlobular bile ducts, which can occur in the presence or absence of inflammation. While classic onion skin fibrosis is pathognomonic of PSC, these lesions are infrequent among PSC patients, particularly in children. Other common histologic findings in humans with PSC are bile ductular proliferation or diminution or absence of interlobular bile ducts (“ductopenia”), degeneration of bile duct epithelium, diffuse infiltration of portal tracts by mononuclear cells and neutrophils, piecemeal necrosis without rosette formation, cholestasis, and fatty change.

The prevalence of PSC in the United States is approximately 1-6 cases per 100,000 population, and the vast majority are Caucasian. Approximately 75% of patients with PSC are men having an average age of approximately 40 years at the time of diagnosis. Most patients with PSC do not exhibit symptoms and are usually diagnosed by the detection of abnormal biochemical tests of liver function on routine blood testing. When symptoms develop they are a result of obstruction to bile flow and include jaundice, itching, right upper quadrant abdominal pain, fever, and chills. Symptoms may also include weight loss and fatigue. Patients may remain asymptomatic for many years despite the presence of advanced disease, and the development of symptoms usually suggests the presence of advanced disease.

Management of this disease in the early stages involves the use of drugs to prevent disease progression. Ursodiol is often used for the treatment of PSC due to improvements in liver biochemistry following initiation of therapy. Despite general biochemical improvement, ursodiol has not been shown to improve transplant-free survival and, at high doses, has been associated with increased risk for serious complications. However, as there are no approved drugs for the treatment of PSC, some physicians treat patients with ursodiol, typically at a dose of 13 to 15 mg/kg/day. Endoscopic and surgical approaches are reserved for the time when symptoms develop. Liver transplantation may ultimately be required and offers the only chance for a complete cure. Indeed, PSC is the fourth leading indication for liver transplant. However, the post-transplant recurrence rate of PSC has been shown to be as high as 20%. Therefore effective treatments are urgently needed to prevent PSC and to delay time to liver transplantation, prevent recurrence following transplantation, and to improve the quality of life for PSC patients. With that goal in mind, novel approaches for treating PSC are described below. These developments are based on the unexpected observation that an SGLT2 inhibitor, remogliflozin etabonate, prevents the progression of PSC disease pathology.

### SUMMARY OF THE INVENTION

The invention relates to treating primary sclerosing cholangitis (PSC) with at least one SGLT2 inhibitor. Methods and compositions associated with the invention improve or maintain clinical outcomes in PSC-afflicted individuals following the administration of an SGLT2 inhibitor, including clinical symptoms such as ascites accumulation, hepatic encephalopathy, development of varices, jaundice, variceal bleeding, cholangiocarcinoma, hepatocellular carcinoma, evidence of cirrhosis, and colorectal cancer.

Abnormal liver function tests can be used to identify PSC patients that can benefit from SGLT2 inhibitor therapy. For example, PSC patients with blood plasma levels greater than the upper limit of normal (ULN) for one or more of Alkaline Phosphatase, Alanine Transaminase, γ-Glutamyl transpeptidase, Aspartate Transaminase, and total Bilirubin can be treated with compositions and methods of the invention, as can PSC patients that present with one or more of liver fibrosis, inflammatory bowel disease, and abnormal liver stiffness.

SGLT2 inhibitors can be administered orally in either an immediate release (“1R”) or a delayed release (“DR”) dosage form, or in a biphasic dosage form containing an IR and DR phase.

### BRIEF DESCRIPTION OF THE FIGURES

FIG. 1A shows liver and biliary pathology in an H&E stained liver section harvested from a wild type mouse. Normal liver histochemistry is observed. PV=branch of the portal vein; HA=branch of the hepatic artery. BD=bile duct. Bar=100 μm.

FIG. 1B shows the presence of multiple portal tracts in an H&E stained liver section harvested from an untreated TIA mouse at 11 weeks. Inflammation is centered around bile ducts, and is accompanied with bile ductular proliferation (multiple bile duct profiles per portal tract; arrows). PV=branch of the portal vein. Bar=100 μm.

FIG. 1C shows the obliteration (oBD; arrowhead) of a portal tract by inflammation in an H&E stained liver section harvested from an untreated TIA mouse at 18 weeks. HA=branch of the hepatic artery. BD=bile duct. PV=branch of the portal vein. Bar=100 μm.

FIG. 1D shows activated immune cells in an H&E stained liver section harvested from an untreated TIA mouse at 18 weeks, that have surrounded, attacked and damaged bile duct epithelial cells (black arrowhead). Bar=100 μm.

FIG. 1E shows the development of onion skin fibrosis of bile ducts in a TIA mouse at 18 weeks of age. Bar=100 μm.

FIG. 2A shows hepatic parenchyma inflammation in an H&E stained liver section harvested from an untreated TIA mouse at 11 weeks. PV indicates portal vein. Bar=500 μm.

FIG. 2B shows biliary inflammation around bile ducts following in an H&E stained liver section harvested from an untreated TIA mouse at 11 weeks. PV indicates portal vein. Asterisks (*) indicate bile ducts. Bar=50 μm.

FIG. 2C shows inflammation at the interface between the hepatic parenchyma and the portal tracts in an H&E stained liver section harvested from an untreated TIA mouse at 11 weeks. PV indicates portal vein. Bar=50 μm.

FIG. 2D shows a decrease in periportal and biliary inflammation in an H&E stained liver section harvested from a TIA mouse at 11 weeks, that received 0.03% Remo in chow, beginning at 4 weeks of age. PV indicates portal vein. Bar=500 μm.

FIG. 2E shows a decrease in proliferation of bile ductules in an H&E stained liver section harvested from an untreated TIA mouse at 11 weeks that received 0.03% Remo in chow, beginning at 4 weeks of age. Asterisks (*) indicate bile ducts. PV indicates portal vein. Bar=50 μm.

FIG. 3 shows a plot of inflammation scores based on the histological examination of H&E stained liver sections harvested from TIA mice at 11 weeks that had been fed either standard chow, or a 0.03% remogliflozin-formulated standard chow, for 7 weeks. Scores were based on the degree of fibrosis, bile ductular proliferation or ductopenia, portal inflammation, lobular inflammation, interface hepatitis, presence of cholangitis, or periductal fibrosis/onion-skinning, as described in Table 1.

### DETAILED DESCRIPTION

Compositions and methods for using an SGLT2 inhibitor for treating individuals afflicted with primary sclerosing cholangitis (PSC) are described herein. Therefore, the invention relates to methods of administering an SGLT2 inhibitor to an individual, typically a human subject, or in other words, a patient, in an amount effective to treat PSC. SGLT2 inhibitors used in methods according to the invention generally, but not necessarily, belong to the gliflozin class of SGLT2 inhibitors. More particular examples of SGLT2 inhibitors that can be used in methods of the invention include, but are not limited to: Canagliflozin (sold under the tradenames, Invokana® and Sulisent®); Dapagliflozin (sold under the tradename, Farxiga®); Empagliflozin (sold under the tradename, Jardiance®); Ertugliflozin (sold under the tradename, Steglatro®); Ipragliflozin (sold under the tradename, Suglat®); Tofogliflozin (currently being developed by Chugai Pharma in collaboration with Kowa and Sanofi); Luseogliflozin (currently being developed by Taisho Pharmaceutical under the tradename Lusefi®); Remogliflozin (currently being developed by Avolynt, Inc. and sold under the tradename Remo® and Remozen®); Sotogloflozin (Also known as LX4211, and currently being developed by Lexicon Pharmaceuticals); Licogliflozin (also know as LIK-066, and currently being developed by Novartis), TFC-039 (currently being developed by Sirona Biochem); Sergliflozin; and salts of the foregoing SGLT2 inhibitors.

SGLT2 is a low affinity, high capacity sodium-glucose cotransporter located mainly in the S1 segment of the proximal tubule of the kidney. SGLT2 inhibition improves glucose clearance from the bloodstream, by increasing urinary glucose excretion. However, SGLT2 protein is also expressed in the central vein and biliary tract of the liver. Therefore, the administration of a SGLT2 inhibitor to a PSC patient can cause the inhibition of SGLT2 activity in liver of a PSC patient, which, in turn, halts the progression of PSC.

Typical PSC-related clinical outcomes include, for example, progression to cirrhosis, liver failure, death and liver transplantation. PSC-related clinical complications include, for example, ascites, hepatic encephalopathy, development of varices, jaundice, variceal bleeding, cholangiocarcinoma, hepatocellular carcinoma, evidence of cirrhosis, and colorectal cancer. A method for treating PSC with a SGLT2 inhibitor in a subject can improve clinical outcomes or clinical complications of PSC.

A patient suffering from PSC who can benefit from SGLT2 inhibitor therapy can have abnormal liver function tests. For example, the patient can have an abnormal ALP test. A PSC patient's blood serum ALP level may be greater than the upper limit of normal (ULN), for example, 1.5 times ULN, 1.6 times ULN, 2 times ULN, 2.5 times ULN, 3 times ULN, 4 times ULN, or a range of 1.5 to 10 times ULN or a range of 3 to 12 times ULN. Other abnormal liver function tests which can be exhibited by a patient suffering from PSC include a tests for blood levels or functions of ALT, GGT, AST, and total bilirubin.

A PSC patient that benefits from SGLT2 inhibitor therapy may also present with liver fibrosis or IBD, or both. Alternatively, a PSC patient undergoing SGLT2 inhibitor therapy may present with liver fibrosis or IBD, or both, but demonstrate normal liver function, based on liver function tests. The IBD can be: Ulcerative colitis (“UC”); Crohn's disease; or Indeterminate, undifferentiated or unclassified IBD (“IBDU”). A patient suffering from PSC who can benefit from SGLT2 inhibitor therapy can also have abnormal liver stiffness. Accordingly, a method according to the invention can be used for treating a PSC patient with a liver stiffness transient elastography (“TE”) score of ≤20 kPa, ≤18 kPa, ≤16 kPa, ≤15 kPa, ≤14 kPa, ≤13 kPa.

An effective amount of an SGLT2 inhibitor for therapy according to method of the invention may be an amount sufficient to reduce, delay or prevent progression of PSC-related clinical complications, liver failure, or death. An effective amount of remogliflozin etabonate also includes any single dosage amount of a SGLT2 inhibitor, which is administered as part of a treatment regimen that includes multiple administrations of the SGLT2 inhibitor. Examples of effective dosage amounts of remogliflozin etabonate can be, but are not limited to, an amount from 5 mg to 2000 mg. Preferred effective dosage amounts of remogliflozin etabonate are, typically, 100, 250 or 400 mg once or twice daily.

An effective amount of remogliflozin etabonate for treating PSC according to the invention can be determined based on various PSC disease metrics. For example, an effective amount of a SGLT2 inhibitor can be an amount that is sufficient to: Maintain, improve, or normalize a clinical disease assessment score; Maintain, reduce, or normalize the level of a marker of liver function or pathology in the subject. An effective amount of remogliflozin etabonate that is administered to a subject can also be sufficient to: Maintain or improve an Ishak fibrosis staging score; Maintain, reduce, or normalize serum ALP; Maintain or improve an Ishak necroinflammatory grading score; Maintain, improve, or normalize an Amsterdam Cholestatic Complaints Score (“ACCS”); Maintain, improve, or normalize 5-D itch scale; Maintain, improve, or normalize the time to progression to cirrhosis, as assessed by a TE score; Maintain, improve, or normalize the time to PSC-related clinical outcomes or clinical complications; Maintain, improve, or normalize a subject's collagen proportional area (“CPA”); Maintain, improve, or normalize Enhanced Liver Fibrosis (“ELF”) score, as assessed by an algorithm using tests for serum concentrations of procollagen-Ill aminoterminal propeptide, tissue inhibitor of matrix metalloproteinase-1 and hyaluronic acid; Maintain, improve, or normalize a liver stiffness score, as assessed by TE or magnetic resonance elastography (“MRE”); or Maintain, improve, or normalize Mayo PSC risk score, or any combination thereof.

As indicated above, an effective dose of a SGLT2 inhibitor can be administered in a unit dose or multiple doses. The dosage can be determined by methods known in the art and can be dependent, for example, upon the individual's age, sensitivity, tolerance and overall well-being. A clinician or pharmacist of ordinary skill can determine appropriate dosing using the guidance provided herein and conventional methods. For example, the levels of a marker, such as, for example, ALP, in the individual being treated can be used as a metric to guide adjustments to an effective dose of a SGLT2 inhibitor to achieve a desired reduction or normalization of the level of the marker.

Examples of modes of administration for a SGLT2 inhibitor include enteral routes, such as through a feeding tube or suppository, and parenteral routes, such as intravenous, intramuscular, subcutaneous, intraarterial, intraperitoneal, intravitreal administration, or orally. For example, a preferred mode of administration for the SGLT2 inhibitor, remogliflozin etabonate, is an oral route for the administration of oral dosage forms of remogliflozin etabonate.

Pharmaceutical compositions of the invention may be prepared by methods known in the pharmaceutical formulation art, for example, see Remington's Pharmaceutical Sciences, 22nd Ed., (Pharmaceutical Press, 2012), which is incorporated herein by reference. In a solid dosage form, a SGLT2 inhibitor may be admixed with at least one pharmaceutically acceptable excipient such as, for example: (a) sodium citrate; (b) dicalcium phosphate; (c) fillers or extenders, such as, for example, starches, lactose, sucrose, glucose, mannitol, and silicic acid; (d) binders, such as, for example, cellulose derivatives, starch, aliginates, gelatin, polyvinylpyrrolidone, sucrose, and gum acacia; (e) humectants, such as, for example, glycerol; (f) disintegrating agents, such as, for example, agar-agar, calcium carbonate, potato or tapioca starch, alginic acid, croscarmellose sodium, complex silicates, and sodium carbonate; (g) solution retarders, such as, for example, paraffin; (h) absorption accelerators, such as, for example, quaternary ammonium compounds; (i) wetting agents, such as, for example, cetyl alcohol, and glycerol monostearate, magnesium stearate; (j) adsorbents, such as, for example, kaolin and bentonite; and (k) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms, including oral dosage forms, may be prepared with coatings and shells, such as enteric coatings and others, as is known in the pharmaceutical art. They may contain pacifying agents, and can be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Liquid dosage forms may be aqueous, may contain a pharmaceutically acceptable solvent as well as traditional liquid dosage form excipients known in the art, which include, but are not limited to, buffering agents, flavorants, sweetening agents, preservatives, and stabilizing agents.

Oral dosage forms according to the invention are typically tablets or capsules. Tablets can be obtained by direct compression of the mixed components of a dosage form, including an effective dosage amount of remogliflozin etabonate, and selected excipients, like cellulose derivatives, metacrylates, chitosan, carboxymethylstarch, or mixtures thereof. For example, a compressed tablet according to the invention, can be prepared by granulating a SGLT2 inhibitor with microcrystalline cellulose and croscarmellose sodium with a water and povidone solution. The resulting granules are dried, milled, and then blended with mannitol, microcrystalline cellulose, and croscarmellose. The blend is lubricated with magnesium stearate and compressed. A compressed IR tablet according to the invention, which, for example, contains an effective dose of 350 mg of remogliflozin etabonate, can be orally administered to a subject to reach a maximum remogliflozin plasma concentration (Cₘₐₓ) of 160 ng/mL at 1 hr post-ingestion, and plasma clearance to 40 ng/mL after 3 hrs. Indeed, Tₘₐₓ for an IR remogliflozin etabonate oral dosage form according to the invention occurs at 1 hour, or less, following ingestion of the dosage form by a subject.

Alternatively, an oral dosage form according to the invention can be soft or hard capsule. For example, a capsule dosage form according to the invention may include SGLT2 inhibitor-layered pellets prepared by coating microcrystalline cellulose spheres with an aqueous suspension containing micronized remogliflozin etabonate, povidone, and purified water. Capsules are typically manufactured from animal-derived gelatin or plant-derived hydroxypropyl methylcellulose (HPMC). The size of a capsule for an oral dosage form of the invention can be any size that is sufficient to contain its effective dose of remogliflozin etabonate and excipient components. For example, the capsule can be a size 5, 4, 3, 2, 1, 0, 0E, 00, 000, 13, 12, 12el, 11, 10, 7, or Su07. Capsules are filled using any suitable techniques.

In various methods according to the invention, an oral dosage form of a SGLT2 inhibitor according to the invention may be an immediate release (“1R”) formulation, or a dosage form designed to release the SGLT2 inhibitor after a period of delay, commonly known as a delayed release (“DR”), extended release, or modified release formulation. Alternatively, it may be appropriate for a SGLT2 inhibitor to partitioned into IR and DR components within a single dosage form.

An IR formulation or formulation component can include one or more hydrophilic materials, or one or more hydrophobic materials, or a combination of hydrophilic and hydrophobic materials. Hydrophilic and hydrophobic materials can be polymers. Examples of a hydrophilic polymers include, but are not limited to: hydroxypropylmethylcellulose, hydroxypropylcellulose, sodium carboxymethylcellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, and potassium pectinate. Examples of hydrophobic polymer that are available for inclusion in an oral dosage form according to the invention include, but are not limited to: Ethyl cellulose; Hydroxyethyl cellulose; An amino methacrylate copolymer; A methacrylic acid copolymer; A methacrylic acid acrylic acid ethyl ester copolymer; A methacrylic acid ester neutral copolymer; A dimethyl-amino-ethyl-methyl-methacrylate-methacrylic acid ester copolymer; A vinyl methyl ether or maleic anhydride copolymer; and Salts and esters thereof. Hydrophobic polymers may also be selected from: A wax, including bees wax, carnuba wax, microcrystalline wax and ozokerite; A fatty alcohol, including cetostearyl alcohol, stearyl alcohol, cetyl alcohol or myristyl alcohol; and A fatty acid ester, including glyceryl monostearate, glycerol monooleate, acetylated monoglycerides, tristearin, tripalmitin, cetyl esters wax, glyceryl palmitostearate, glyceryl behenate, and hydrogenated castor oil.

DR dosage forms can be tablets, filled capsules or remogliflozin etabonate-layered pellets, which are coated with a DR coating, also known as an enteric coating. A DR coating protects an oral dosage form according to the invention from the harsh, acidic environment of the stomach, so that release of the effective dose of remogliflozin etabonate is delayed until the dosage form reaches the small intestine. Any DR coatings of oral dosage forms of the invention are applied to a sufficient thickness such that the entire coating does not dissolve in the gastrointestinal fluids at pH below about 5. A DR coating typically includes a polymer, such as an aqueous dispersion of anionic polymers with methacrylic acid as a functional group like the product sold as Eudragit® L30D-55 (Evonik Industries). A DR coating can also optionally include a plasticizer, such as triethyl citrate, an anti-tacking agent, such as talc, and a diluent, such as water. For example, a coating composition used to coat and oral dosage form of the invention can contain about 42% (wt %) of an aqueous dispersion of anionic polymers with methacrylic acid as a functional group; about 1.25 wt % of a plasticizer; about 6.25 wt % of an anti-tacking agent; and about 51 wt % of a diluent. Another example of a coating compostions for an oral dosage form of the invention, particularly when a large-scale preparation is preferred, an appropriate amount of an anionic copolymer based on methacrylic acid and ethyl acrylate, such as Eudragit© L100-55, is used in place of Eudragit© L30D-55. Conventional coating techniques such as spray or pan coating are employed to apply coatings. For example, a coating composition can be applied to capsules of the invention by using a Procept® coating machine and Caleva® mini coater air suspension coating machine to coat the capsules until they experience a 10% to 18% weight gain.

### Examples

The following Examples describe the utilization of a murine model of Primary Biliary Cholangitis (“PSC”) to assess the effectiveness of a treatment regimen based on the oral administration of remogliflozin etabonate. The murine PSC model is based on mice that are deficient for the expression of tumor necrosis factor alpha (“TNFα”), interleukin 10 (“IL-10”), and activation-induced cytidine deaminase (“AICDA”). As the mice are deficient in TNF, IL-10, and AICDA, they are referred to, herein, as “TIA” mice.

TIA mice can exhibit ulcerative colitis (“UC”)-like symptoms and pathology, as well as develop inflammation of the liver and biliary tract that, histologically, resembles PSC in humans. Moreover, as AICDA is required for immunoglobulin (“Ig”) class switching, TIA mice lack IgG and IgA, a phenotype analogous to humans with hyper-IgM syndrome. Therefore, with the combination of AICDA deficiency with the risk factors associated with TNFα and IL-10 deficiencies, TIA mice also develop liver and biliary inflammation reminiscent of PSC symptoms in humans. Accordingly, the TIA model is useful for investigating mechanisms that act early in PSC pathogenesis, as well as treatments that can prevent progression to PSC.

Example 1. Orally-administered remogliflozin etabonate reduces inflammatory cell infiltration, bile ductular proliferation, and interface hepatitis in TIA mice. TIA mice were created by first breeding TNFα knock out (“KO”) C57BL/6 mice, (strain B6.129S-Tnf^{tm1Gkl}/J, stock #005540, Jackson Laboratories, Bar Harbor, Me.) with IL-10 KO mice (strain B10.129P2(B6)-IL10^{tm1Cgn}/J, Stock No. 002251, Jackson Laboratories) to produce a population of mice that were deficient in TNFα and IL-10. Because mice with a TNFα−/− and IL10−/− genotype spontaneously develop inflammatory bowel disease (“IBD”) (Hale 2012), a condition associated with poor breeding success (Nagy 2016), the mice needed for further breeding to generate an AICDA population, were generated by breeding offspring with a TNFα−/− and and IL10+/− genotype with AICDA−/− mice, which were obtained from Dr. Tasuku Honjo (Muramatsu 2000)), to produce a population of TNFα−/−, IL10−/−, and AICDA+/−(“TI-hetA”) males and females. In turn, TI-hetA pairs were bred to generate populations that were 25% TIA mice, and 50% non-colitis-susceptible TI-hetA littermates that could be used as control populations. All populations were exposed to the same environment from birth. The mice were housed in polycarbonate micro-isolator cages, in individually ventilated racks, under barrier conditions that excluded all known pathogens, including Helicobacter pylori and Norovirus. Mice had ad libitum access to water, and to a standard diet (PicoLab Mouse Diet 20/5058, LabDiet, St. Louis, Mo., USA).

At four (4) weeks of age, TIA (40) and TI-hetA (22) mice were randomized into experimental groups that received either a standard diet (20 TIA and 12 het), or a standard diet formulated 0.03% remogliflozin etabonate (20 TIA and 10 het) (Avolynt Inc., USA). The mice were maintained on this diet for seven (7) weeks. Body weights were obtained three (3) times, weekly, to assess the general health of mice, and to track the development of inflammatory bowel disease (IBD). Glycosuria in the experimental groups was assessed by applying freshly voided urine directly to the glucose test patch on an Accutest® URS-10 urinary reagent test strips (Jant Pharmaceutical Corp., Encino, Calif., USA). Mice were humanely euthanized before reaching the experimental endpoint, of eleven (11) weeks of age, if they lost>15% body weight, or developed rectal prolapse.

To characterize biliary lesions in TIA mice at the end of the 7 week treatment period, liver tissue was obtained from the remogliflozin-treated, and untreated groups, for histologic examination. The excised liver tissue was fixed in Carnoy's fixative solution, and processed into paraffin blocks. The paraffin blocks were sectioned, and stained with Hematoxylin and eosin (H&E) for pathologic analysis. The H&E-stained sections were scored by an American Board of Pathology-certified pathologist. The pathologist was blinded to mouse identity, and used an inflammation scoring system that was based on a modification of previously described scoring systems, and in accordance with guidelines suggested by the International PSC Study Group (“IPSG”). Inflammation scores were based on the degree of fibrosis, bile ductular proliferation or ductopenia, portal inflammation, lobular inflammation, interface hepatitis, presence of cholangitis, or periductal fibrosis/onion-skinning. Table 1 summarizes the scoring system used to assess the tissues in this study.

| Histologic | | |
|---|---|---|
| Parameter | Score | Description |
| Histologic stage | 1 | Normal to slight enlargement |
| of fibrosis, | | of portal tracts |
| (Ludwig 1986) | 2 | Portal expansion and periportal fibrosis |
| | 3 | Septal and/or bridging fibrosis |
| | 4 | Cirrhosis |
| Ductular | 0 | Absent |
| proliferation | 1 | Rare |
| | 2 | Present in 5-30% of portal areas |
| | 3 | Present in 30-90% of portal areas |
| | 4 | Expansion of >90% of portal areas |
| | | with numerous duct profiles |
| Ductopenia | 0 | Absent |
| | 1 | Absence of interlobular and septal |
| | | bile duct in >50% of portal areas |
| Degree of portal | 0 | Absent |
| inflammation | 1 | Mild (some or all portal areas) |
| | 2 | Moderate (some or all portal areas) |
| | 3 | Severe (all portal areas) |
| Intralobular | 0 | Absent |
| inflammation | 1 | Mild (≤2 foci per 10X field) |
| | 2 | Moderate (3-5 foci per 10X field) |
| | 3 | Severe (>5 foci per 10X field) |
| Hepatocellular | 0 | Absent |
| mitoses | 1 | Present |
| Interface | 1 | Absent |
| hepatitis | 2 | Focal inflammation present around |
| (“piecemeal | | a minority of portal triads |
| necrosis”) | 3 | Mild to moderate inflammation |
| | | present around most portal triads |
| | 4 | Moderate inflammation continuous |
| | | around <50% of tracts |
| | 5 | Moderate to severe inflammation |
| | | continuous around >50% of tracts |
| Cholangitis | 0 | Absent |
| (inflammation in | 1 | Present |
| bile duct lumen) | | |
| Onion-skinning | 0 | Absent |
| (fibrosis | 1 | Present |
| around bile | | |
| duct) | | |

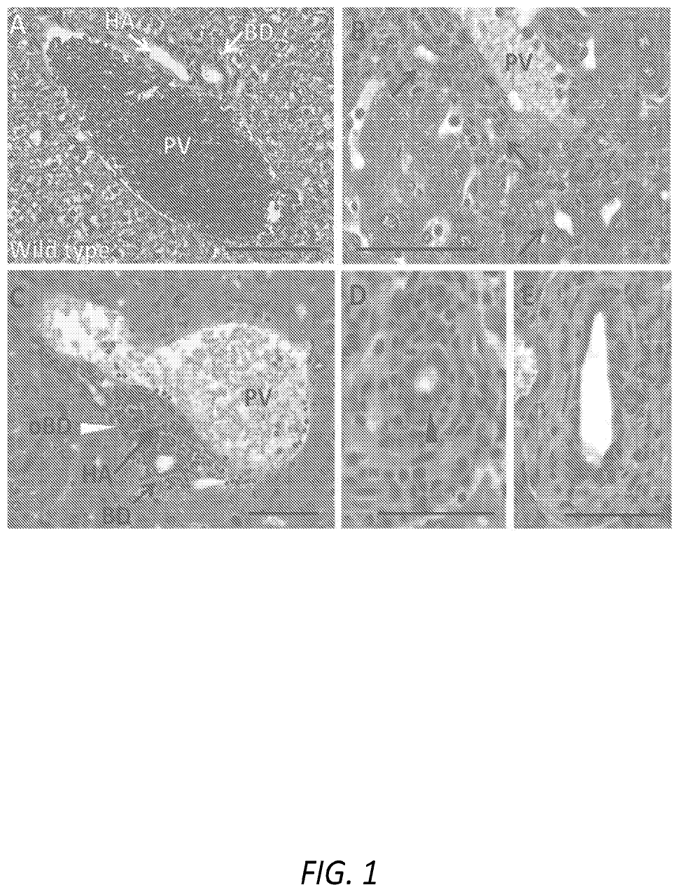

At 11 weeks, the livers of untreated TIA mice generally exhibited PSC-like histologic lesions, including liver and biliary lesions, bile ductular proliferation, and interface hepatitis. See FIGS. 1A-B. Relatively few mice, however, formed major fibrotic lesions, such as onion skin fibrosis of bile ducts or ductopenia by 11 weeks, though such lesions were observed at 18 weeks (FIGS. 1C-E), and could be observed as early as 6 weeks in some TIA mice (data not shown). There were also relatively few mice that had developed cirrhosis, though macronodular cirrhosis was grossly observed in one TIA mouse at 28 weeks, before requiring euthanasia for weight loss (data not shown).

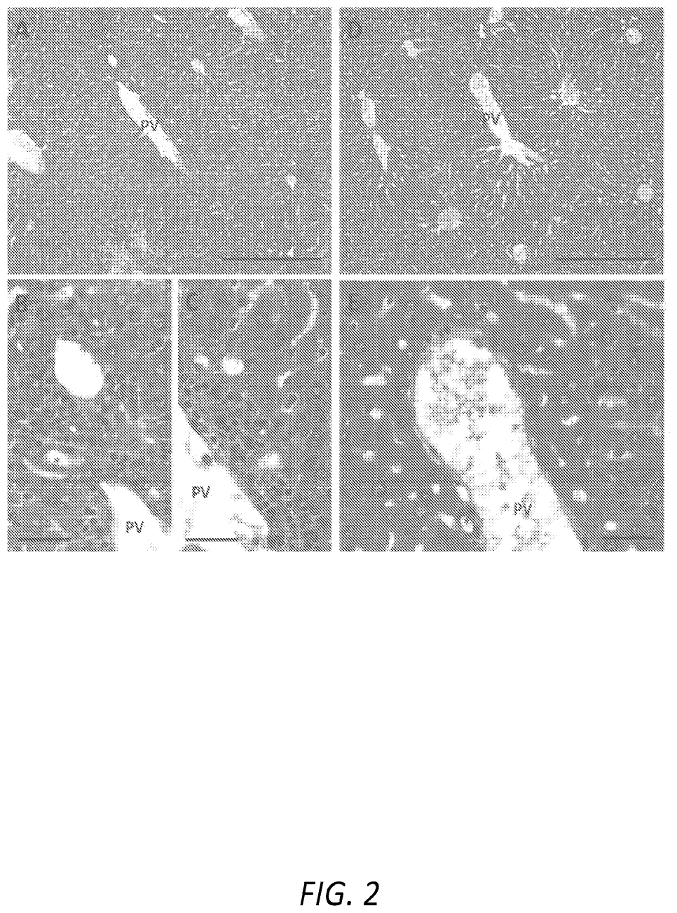

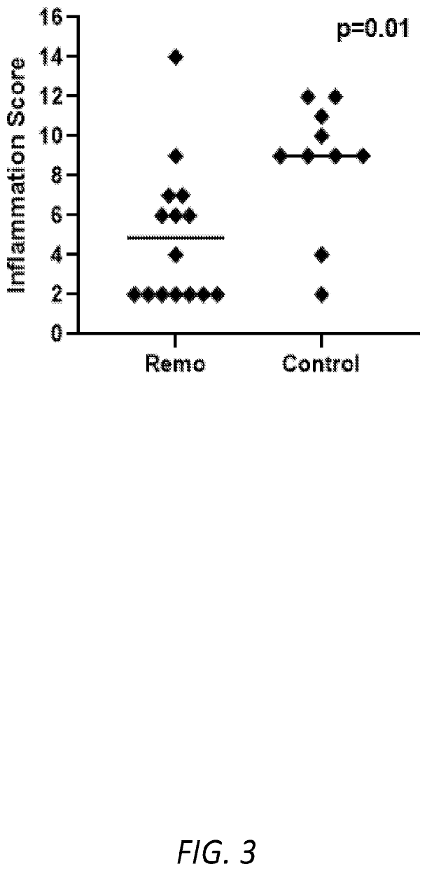

TIA mice, which fed on the remogliflozin etabonate-formulated diet for 7 weeks experienced markedly less development and progression of liver and biliary disease in comparison to TIA mice that remained on a standard diet. More specifically, the remogliflozin-fed TIA mice developed less inflammation at the interface between the hepatic parenchyma and the portal tracts (FIG. 2C), and periportal and biliary regions (FIG. 2D). The remogliflozin-fed TIA mice also experienced less proliferation of bile ductiles in comparison with untreated TIA mice. See FIG. 2E.

While there was no statistical difference in the number of TIA mice that required early euthanasia in the Remo group versus the control group in this study, survival curves of untreated TIA mice suggest a linear rate of death from 5-20 wks (n=90). Therefore, while not statistically significant, the trend toward decreased early death in the Remo group suggests that larger group sizes may uncover survival differences that this small study was not powered to detect.

Example 2. TIA mice demonstrate serologic evidence of of liver and/or biliary injury in TIA mice. A serum biochemical profile of TIA mice at 11 weeks was performed. Blood was drawn from euthanized animals into lithium heparin tubes, and a panel of analytes, including total protein, albumin, serum alkaline phosphatase (AP), alanine aminotransferase (ALT), and total bilirubin were measured using a Heska Dry Chem 7000 analyzer. Serum aspartate aminotransferase (AST) was measured in a separate test. In 50% of the mice, elevated levels of AP, ALT, and AST, which were at least 1.5× the upper limit of normal—levels considered to be indicative of cholestasis/liver damage, were detected. Histological analysis at 11 weeks, as described in Example 1, revealed considerable biliary and hepatic inflammation is present, but relatively little fibrosis. These serum biochemistry data are also suggestive of autoimmune hepatitis.

What is claimed is:

1. A method for treating primary sclerosing cholangitis (PSC), comprising administering an SGLT2 inhibitor, or a salt thereof.

2. The method according to claim 1, wherein the SGLT2 inhibitor, or a salt thereof, is administered orally.

3. The method according to claim 2, wherein the SGLT2 inhibitor, or salt thereof, is formulated as an oral dosage form.

4. The method according to claim 3, wherein the oral dosage form comprises:
a) SGLT2 inhibitor, or salt thereof,
b) at least one hydrophilic or hydrophobic material, or both, and
c) at least one pharmaceutically acceptable excipient.

5. The method according to claim 4, wherein the at least one hydrophilic or hydrophobic material is a polymer.

6. The method according to claim 3, wherein the oral dosage form is a tablet or a capsule.

7. The method according to claim 3, wherein the SGLT2 inhibitor, or a salt thereof, is present in an amount from 1 mg to 2000 mg.

8. The method according to claim 4, wherein the at least one hydrophilic or hydrophobic polymer is a hydrophilic polymer selected from the group consisting of hydroxypropyl methylcellulose, hydroxypropyl cellulose, sodium carboxymethyl cellulose, carboxymethyl cellulose calcium, ammonium alginate, sodium alginate, potassium alginate, calcium alginate, propylene glycol alginate, alginic acid, polyvinyl alcohol, povidone, carbomer, potassium pectate, and potassium pectinate.

9. The method according to claim 4, wherein the at least one hydrophilic or hydrophobic polymer is a hydrophobic polymer selected from the group consisting of ethyl cellulose, hydroxyethyl cellulose, amino methacrylate copolymer, methacrylic acid copolymers, methacrylic acid acrylic acid ethyl ester copolymer, methacrylic acid ester neutral copolymer, dimethylaminoethylmethyl methacrylate-methacrylic acid ester copolymer, vinyl methyl ether/maleic anhydride copolymer, and salts and esters thereof.

10. The method according to claim 4, wherein the at least one hydrophilic or hydrophobic polymer is a hydrophobic polymer selected from the group consisting of a wax, a fatty alcohol, and a fatty acid ester.

11. The method according to claim 10, wherein:
A. the wax is bees wax, carnauba wax, microcrystalline wax or ozokerite;
B. the fatty alcohol is cetostearyl alcohol, stearyl alcohol, cetyl alcohol or myristyl alcohol; and
C. the fatty acid ester is glyceryl monostearate, glycerol monooleate, acetylated monoglycerides, tristearin, tripalmitin, cetyl esters wax, glyceryl palmitostearate, glyceryl behenate or hydrogenated castor oil

12. The method according to claim 4, wherein the at least one pharmaceutically acceptable excipient is a binder, a filler, a lubricant, a preservative, a stabilizer, an anti-adherent, a glidant, or a combination thereof.

13. The method according to claim 4, comprising the excipients: Povidone; Microcrystalline cellulose; Croscarmellose cellulose; and Magnesium stearate.

14. The method according to claim 3, wherein the oral dosage form is an enterically-coated tablet.
